(19) United States
(12) Patent Application Publication
(10) Pub. No.: US 2005/0075612 A1
(43) Pub. Date: Apr. 7, 2005

(54) Parylene coated fluid flow regulator

(75) Inventors: Yann-Per Lee, Vernon Hills, IL (US); Terry L. Kochersperger, Spring Grove, IL (US)

Correspondence Address: BAXTER INTERNATIONAL INC., DF2-2E, One Baxter Parkway, Deerfield, IL, 60015-4633, US

(73) Assignee: Baxter International Inc.

(21) Appl. No.: 10/678,568

(22) Filed: Oct. 3, 2003

(51) Int. Cl. A61M 5/00

(52) U.S. Cl. 604/246

(57) ABSTRACT

The present invention is directed to a device for regulating the flow of a fluid to be delivered intravenously and comprises, in a particular embodiment, a parlyene-coated top (18) having an inlet and a bottom (20) having an outlet, wherein the top and bottom are rotatably connected and define a housing (12). The inlet and the outlet define a fluid passage through the housing (12). The device further comprises a diaphragm (14) and a diaphragm holder (16) disposed in the housing (12). The diaphragm (14) and the diaphragm holder (16) regulate the flow of fluid in the fluid passage in response to the rotation of the top (18) relative to the bottom (20). The device is characterized in having a medium static turning torque and a medium dynamic turning torque.

### TECHNICAL FIELD

The invention relates generally to a fluid flow regulator device, particularly where one or more components is coated with parylene. More specifically, the present device relates to an intravenous fluid flow regulator for controlling the flow of a medical fluid, including blood, in an intravenous delivery system and where the device has at least one component coated with parylene.

### BACKGROUND OF THE INVENTION

In the medical field, medical fluids or solutions are commonly administered to patients by intravenous (I.V.) techniques. The medical fluid is usually contained within an I.V. bag, which is suspended above the patient by an I.V. pole. An I.V. tubing line connects the I.V. bag of medical fluid to the patient through an I.V. needle or catheter inserted into the patient's venous system. The medical fluid flows from the elevated I.V. bag into the patient due to the force of gravity. Medical fluids can also be administered to a patient by an I.V. infusion pump connected to an I.V. tubing line. Devices that utilize these types of I.V. administration techniques are termed I.V. administration sets.

Frequently, the rate in which the medical fluid is administered to the patient must be controlled to provide proper medical treatment. Accordingly, the medical fluid is administered to the patient over an extended period of time rather than being entirely infused into the patient immediately. Of course, various medical treatments and various medical fluids may require different rates of I.V. fluid administration. The rate of I.V. fluid administration is dependant, in part, on the fluid pressure in the I.V. administration set.

Various devices and techniques have been utilized to control fluid pressure in the I.V. administration set and the corresponding fluid flow rate of the medical fluid to the patient. A clamp, for example, may be placed on the I.V. tubing line to partially restrict the flow of fluid through the tubing. However, the clamping force applied by the clamp, the amount of tubing restriction, and the control of the fluid flow rate are subject to considerable variability. One device that purports to control the rate of fluid flow is disclosed in U.S. Pat. No. 4,343,305 to Bron, which is incorporated by reference. Another device that controls the rate of fluid flow is disclosed in U.S. Pat. No. 5,520,661, which has the same assignee as the present invention and is incorporated by reference.

At least one problem with the prior art fluid flow regulators is that those regulators are generally assembled using liquid silicone coating for lubrication. Over time, silicone migrates to neighboring device components, which potentially degrades the regulator's functionality. That change in functionality may, in turn, adversely affect the device's ability to deliver a constant fluid flow, particularly over a varied hydrostatic head height. Another problem with using liquid silicone is that silicone cannot easily penetrate crevices or cover surface irregularities in the device. Still yet another problem with the use of liquid silicone is that organic solvents such as hydrofluoroether are sometimes used in order to dilute the viscous silicone to make the silicone easier to use in the coating process. This leads to increased costs and potential environmental concerns.

The present invention is designed to solve these and other problems.

### SUMMARY OF THE INVENTION

The present invention provides an intravenous fluid flow regulator device for controlling the I.V. administration of medical fluid to a patient. The fluid flow regulator comprises a housing having two components, a top and a bottom. At least one of the components is coated with parylene. The top has an inlet, and the bottom has an outlet. Together, the inlet and outlet define a fluid passage through the housing. The device further comprises a flexible diaphragm positioned within the housing. The diaphragm is adapted to be sealingly engaged to a diaphragm holder, which is also located within the housing. The diaphragm holder may also be coated with parylene.

It is understood that both the foregoing general description and the following detailed description, including the drawings, are exemplary and explanatory and are intended to provide further explanation of the invention as defined by the claims.

### BRIEF DESCRIPTION OF THE DRAWINGS

FIG. 1 is an elevational, partial cross-sectional view showing a fluid flow regulator in accordance with the present invention.

FIG. 2 is an elevational, full cross-sectional view of the fluid flow regulator of FIG. 1.

FIG. 3 is a perspective view of the bottom of the fluid flow regulator of FIGS. 1 and 2.

FIG. 4 is a top view of FIG. 3.

FIG. 5 is an enlarged top view of a portion of FIG. 4 showing a rib in greater detail.

FIG. 6 is an enlarged side elevational view of a portion of FIG. 4 taken along line 6-6 showing the ribs in greater detail.

FIG. 7 is a cross-sectional view of a rib taken along line 7-7 of FIG. 4.

FIG. 8 is a cross-sectional view of the fluid flow regulator showing the fluid flow path through the regulator.

FIG. 9 is a graphical representation of the medium turning torques for certain embodiments of the present invention.

### DETAILED DESCRIPTION

While this invention is susceptible of embodiments in many different forms, and will herein be described in detail, preferred embodiments of the invention are disclosed with the understanding that the present disclosure is to be considered as exemplifications of the principles of the invention and are not intended to limit the broad aspects of the invention to the embodiments illustrated.

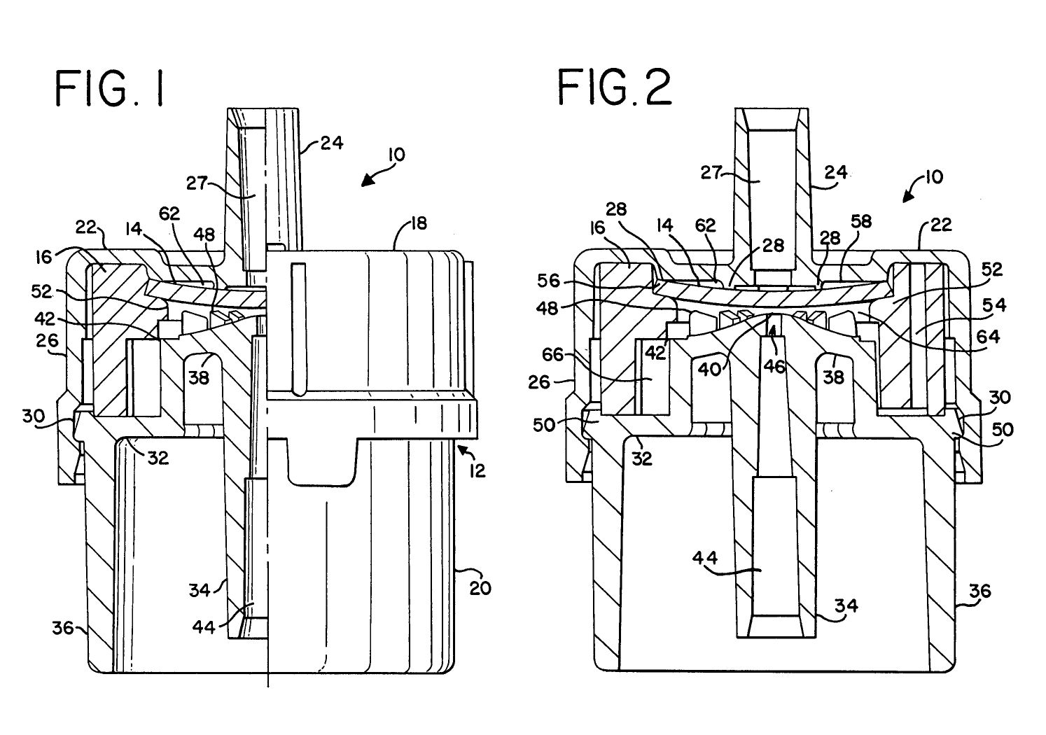

FIG. 1 shows a elevational, partial cross-sectional view of a fluid flow regulator device 10 made in accordance with the present invention. FIG. 2 is a full cross-sectional view of the fluid flow regulator device 10 shown in FIG. 1. In one embodiment, the fluid flow regulator 10 comprises a housing 12, which is constructed from a top 18 rotatably connected to a bottom 20, and a flexible diaphragm 14. The flexible diaphragm 14 is positioned within a diaphragm holder 16. The diaphragm 14 and diaphragm holder 16 are positioned within the housing 12, as described in greater detail below.

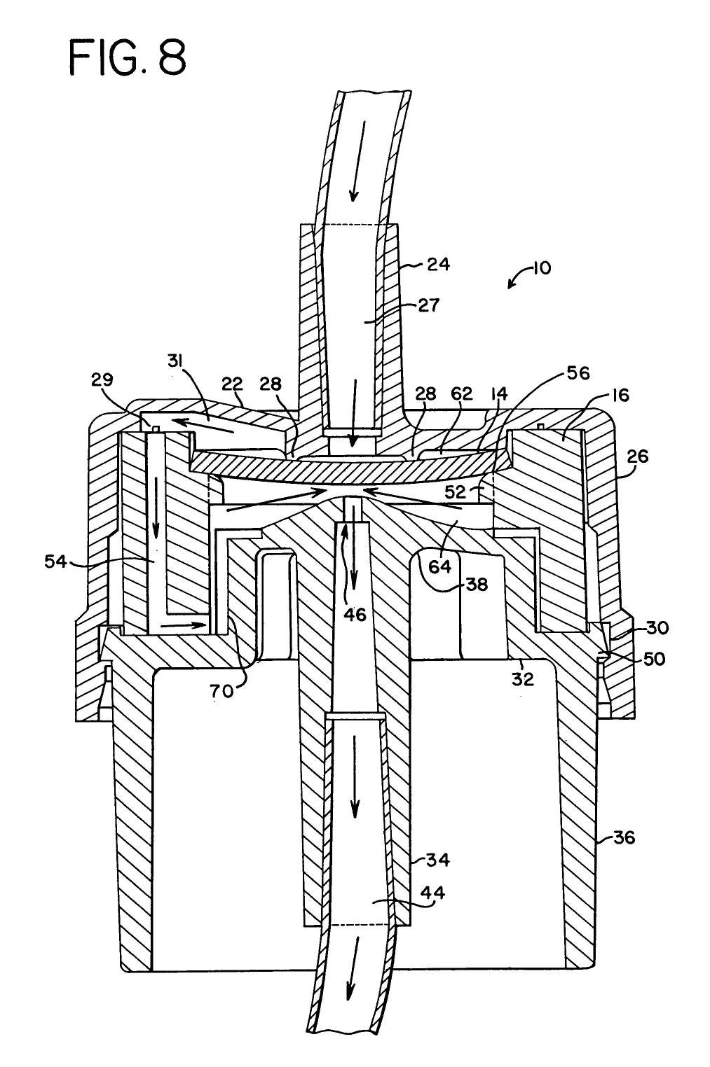

The top 18 comprises a top wall 22, a fluid inlet 24, and a top side wall 26. The top wall 22 is approximately circular in shape. The fluid inlet 24 is connected to and extends upwardly from the top wall 22 and provides an inlet fluid passage 27 through top wall 22. The fluid inlet 24 is connected to the top wall 22 at about the center of the top wall 22. A protrusion 28 is provided on the interior side of the top wall 22. The protrusion 28 contacts the diaphragm 14 to bias the diaphragm 14 towards the bottom 20 of the housing 12. The top wall 22 defines an arcuate capillary groove 29 that is shown more clearly in FIG. 8. The arcuate capillary groove 29 extends around the center of top wall 22 through an arc of less than 360 degrees. Preferably, the capillary groove 29 extends through an arc of about 270 degrees. The capillary groove 29 provides a fluid flow restriction, which causes a fluid pressure drop as fluid flows through the capillary groove 29.

The top wall 22 further defines a top fluid channel 31 that extends from about the center of the top wall 22 to the capillary groove 29. As shown in FIG. 2, the top side wall 26 extends downwardly from top wall 22 around the periphery of the top wall 22 and in an opposite direction from the fluid inlet 24. The top side wall 26 provides a locking recess 30 for engaging the bottom 20 when the top 18 and the bottom 20 are rotatably connected together to comprise the housing 12.

The bottom 20 comprises a bottom wall 32, a fluid outlet 34, and a bottom side wall 36. The bottom wall 32 is approximately circular in shape and has a raised portion 38. The raised portion 38, comprising a center 40 and an edge 42, extends upwardly from the bottom wall 32. The center 40 extends further from the bottom wall 32 relative to the edge 42. The raised portion 38 slopes downwardly from the center 40 to the edge 42. The fluid outlet 34 is connected to the raised portion 38 at the center 40 and extends downwardly in an opposite direction from the raised portion 38. The fluid outlet 34 provides an outlet fluid passage 44 through the bottom wall 32 via an outlet opening 46 in the raised portion 38.

The bottom side wall 36 extends downwardly from bottom wall 32 around the periphery of bottom wall 32 and in the same direction as the fluid outlet 34. The bottom side wall 36 provides a locking ridge 50 for engaging the top 18, particularly the locking recess 30, when the top 18 and the bottom 20 are rotatably connected together.

The diaphragm holder 16 is disposed in the housing 12 proximate to the bottom and has an annular ring shape and an annular ledge 52. The annular ledge 52 extends from the diaphragm holder 16 towards the inside of the annular ring shape of the diaphragm holder 16. The annular ledge 52 sealingly engages the diaphragm 14. The diaphragm holder 16 has a bypass fluid channel 54 through at least a portion of the diaphragm holder 16. The bypass fluid channel 54 permits fluid to flow around the diaphragm 14, as described below.

The diaphragm 14 and the diaphragm holder 16 are each preferably constructed of a flexible, resilient material, though not necessarily of the same material. The diaphragm 14 is generally circular in shape and has a fluid sealing edge 56 that contacts the annular ledge 52 of the diaphragm holder 16 to form a fluid-tight seal. The diaphragm 14 also contacts the protrusion 28 of the top wall 22 of the top 18 on a side of the diaphragm 14 that is opposite that of the sealing edge 56.

According to an embodiment of the present invention, the top 18 is coated with parylene. Parylene is the common name for a particular polymer series, which includes parylene N, parylene C, and parylene D as shown below.

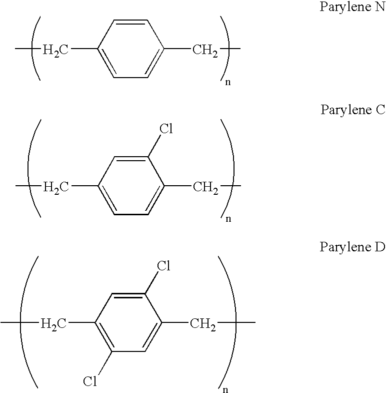

Parylene provides a pinhole-free barrier to moisture, chemicals, biofluids and biogases, though it may have small, measurable permeabilities to water molecules and some common gases. Parylene also has excellent dry film lubricant characteristics, which can be quantified by measuring its coefficient of friction. The top 18 is preferably coated with parylene, preferably parylene N, with the coating thickness being from about 0.10 microns to about 3.0 microns. It is understood by one of ordinary skill that 1 micron is 1.0×10⁻⁶ meters. In another embodiment, the diaphragm holder 16 is coated with parylene, preferably parylene N, with the coating thickness being from about 0.10 microns to about 3.0 microns. Even more preferably, where a component is coated with parylene, the thickness of the coating is from about 0.50 microns to about 2.0 microns. In yet another embodiment, both the top 18 and the diaphragm holder 16 are coated with about 1.4 microns of parylene, preferably parylene N.

The use of a parylene coating on the top 18, and preferably a parylene coating on the diaphragm holder 16, resulted in the fluid flow regulator device 10 having a more constant flow rate regardless of the hydrostatic head height, particularly when the head height was varied from about thirty inches to about sixty inches. The flow compensation through the device is governed by a disk-shaped, elastomeric diaphragm, and the diaphragm's ability to flex to allow a constant ΔP pressure differential to form across the restrictive portion of the flow regulator. When the head height of IV fluid causes an internal device pressure differential greater than that needed to push the disk against the fluid outlet, the flow is briefly stopped, causing the ΔP across the resistive portion of the device to stabilize. Thus, this ΔP level remains virtually constant for cases when the external head height pressures acting on the device are greater than that needed to cause the disk to shut off flow through the outlet portion.

The use of a parylene coating also includes the benefit of eliminating silicone migration, as may be observed when a liquid silicone coating is used. The undesirable migration of silicone may adversely affect the performance of the fluid flow regulator device 10, including potentially changing the device's components' mechanical properties. In this sense, coating at least one of the components of the fluid flow regulator device 10 with parylene enables a more robust device performance feature of head height compensation than a conventional, migratory liquid silicone coating.

Another benefit of using parylene over liquid silicone is that the parylene coating process is essentially solvent free. Liquid coating of silicone, on the other hand, usually involves the use of organic solvents, such a hydrofluoroether, to dilute the viscous silicone. Thus, the use of parylene instead of silicone potentially minimizes chemical exposure and may be more environmentally sound.

Another benefit of the parylene coating is that it imparts a unique turning torque profile for the fluid flow regulator device 10. The turning torque profile is represented by the data in Table 1.

TABLE 1

| | COATING | | TURNING TORQUE RANGE | | |
|---|---|---|---|---|---|
| | | Diaphragm | (in.-oz.) n = 20 | | |
| Sample | Top | Holder | Static | Dynamic | Overall |
| A | Silicone | Silicone | 36-56 | 10-18 | 10-56 |
| B | Parylene N | Parylene N | 28-38 | 20-32 | 20-38 |
| C | None | Parylene N | 24-136 | 22-110 | 22-136 |
| D | Parylene N | None | 32-58 | 24-37 | 24-58 |

Four different embodiments of the fluid flow regulator device 10 were tested as per Table 1. Sample A had its top 18 and diaphragm holder 16 coated with liquid silicone. Sample B has both its top 18 and diaphragm holder 16 coated with parylene N. Sample C had its top 18 uncoated and its diaphragm holder coated with parylene N. Sample D had its top 18 coated with parylene N and its diaphragm holder 16 uncoated. Each parylene coating was about 1.4 microns thick, though the thickness ranges may vary as described above.

The parylene coating was applied using a vapor deposition process, which is known to those skilled in the art and one method of which is briefly described. A dimer of di-para-xylylene is heated to about 150° C. at about 1.0 torr to convert the dimer to a vapor. The vapor yields the para-xylyene monomer, which is pyrolized at about 680-690° C. at about 0.5 torr. The monomer is polymerized, and the device was coated at ambient temperature and about 0.1 torr. The vapor deposition of parylene is preferred over the liquid coating of silicone because the vapor can more easily cover an irregular surface, as well as penetrate into crevices and over contours that the liquid cannot.

Before the turning torque was measured, each sample underwent gamma sterilization at a dosage of about 26.9 to about 28.6 KGy and was stored under conditions of 135° F. at a relative humidity of 50% for seven days. To calculate the turning torque range, a sample size of twenty measurements was taken. One purpose of measuring the range was to determine the static and dynamic turning torque required to rotate the top 18 relative to the bottom 20. A torque meter in combination with a top cap torque fixture was used to measure the turning torque. Three separate torque meters were used, with the torque ranges being 048 in.-oz. 0-96 in.-oz. and 0-192 in.-oz. which are commercially available from the Snap-On Tools Corporation.

Before each measurement, the meter's reading scale and peak indicator were calibrated and zeroed. The bottom 20 was then inserted into the torque fixture and applied to the torque meter. The sample was then turned counterclockwise for approximately one revolution over thirty seconds. As the sample was being turned, the peak indicator recorded the sample's static torque. The average fluctuation of the torque value during turning was recorded as the sample's dynamic torque. One turn yielded static and dynamic torque values.

As stated above, another benefit of the parylene coating is that it imparts a unique turning torque profile for the fluid flow regulator device 10, which may generate a more stable and consistent fluid flow profile than if a conventional silicone coating were used. The accuracy of the device's flow rate is partially controlled by the compression property of diaphragm holder. A more compressible diaphragm holder may lead to under-infusion during IV administration. A diaphragm holder coated with liquid silicone may potentially exhibit varied mechanical properties depending on the amount of liquid silicone coating that may have soaked into the diaphragm holder. Using parylene, instead, modifies only the surface property of the diaphragm holder and does not significantly change the bulk property of diaphragm holder. The parylene-coated diaphragm holder thus may display a more stable and consistent fluid flow profile than a silicone-coated diaphragm holder.

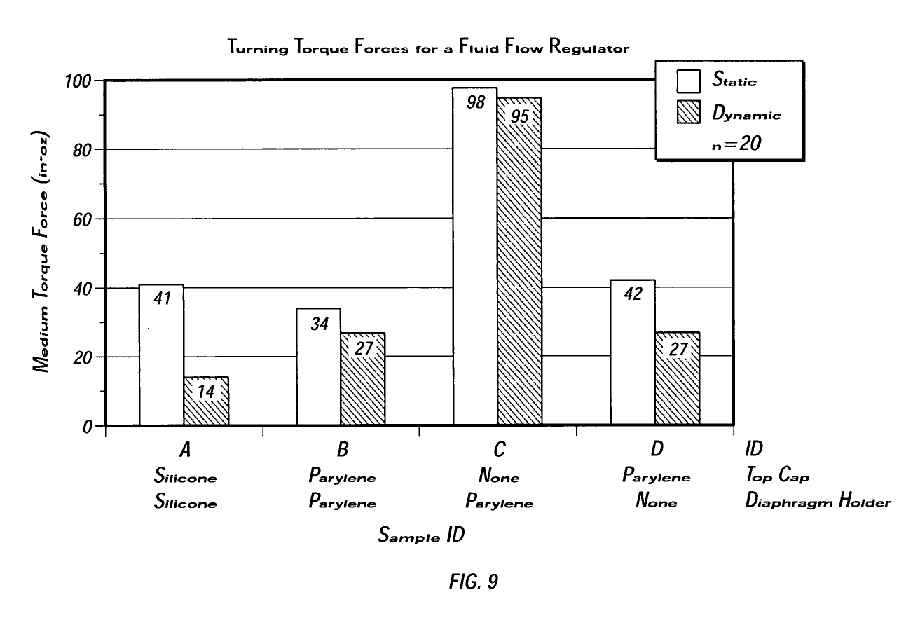

The medium static turning torque and the medium dynamic turning torque for each sample, together with their respective aggregate sums and differences, are shown in FIG. 9, as well as in Table 2.

TABLE 2

| | COATING | | MEDIUM TURNING TORQUES | | | |
|---|---|---|---|---|---|---|
| | | Diaphragm | (in.-oz.) n = 20 | | | |
| Sample | Top | Holder | Static | Dynamic | Sum | Difference |
| A | Silicone | Silicone | 41 | 14 | 55 | 27 |
| B | Parylene | Parylene N | 34 | 27 | 61 | 7 |
| | N | | | | | |
| C | None | Parylene N | 98 | 95 | 193 | 3 |
| D | Parylene | None | 42 | 27 | 69 | 15 |
| | N | | | | | |

The fluid flow regulator device 10 that was labeled as Sample B with its top 18 and diaphragm holder 16 coated with parylene N exhibits a unique and desirable turning torque profile of having lower medium static torque than all the other samples but a moderate medium dynamic torque relative to Sample A, which had its top 18 and diaphragm holder 16 coated with liquid silicone. Further, each sample exhibited a medium dynamic turning torque less than its static dynamic turning torque, meaning that the static dynamic turning torque is the upper limit for both medium turning torques. As applied to Sample D, it shows a medium static turning torque of about 42 in.-oz. The sum of the torque values for Sample D will thus be less than about 84 in.-oz. The reported value in Table 2 of that sum for Sample D, 69 in.-oz., is in accord.

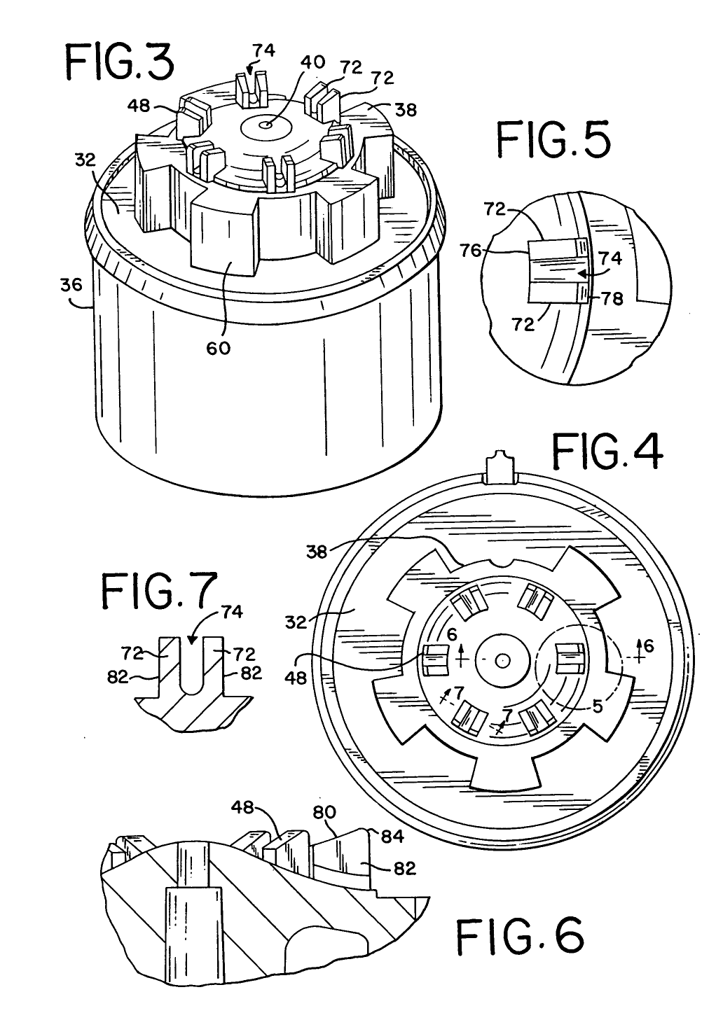

One embodiment of the fluid flow regulator device 10 is shown in FIG. 2. The top 18 and the bottom 20 are rotatably connected together to form the housing 12. The top 18 and the bottom 20 are preferably concentrically aligned such that fluid inlet 24 and fluid outlet 34 are also concentrically aligned. The locking ridge 50 on the bottom 20 engages the locking recess 30 on the top 18 to selectively and lockingly connect the top 18 and the bottom 20 together. The top 18 and the bottom 20 are locked together to prevent movement along a central axis, though they can be rotated relative to each other around the central axis. The flexible, resilient diaphragm holder 16 is positioned within the housing 12 and between the top 18 and the bottom 20, and is preferably concentrically aligned with the top 18 and the bottom 20. When the top 18 is rotated relative to the bottom 20, the diaphragm holder 16 preferably rotates relative to the top 18 but not relative to the bottom 20. To prevent the diaphragm holder 16 from rotating relative to bottom 20, tabs 60 are disposed on the bottom 20 and engage the tab recesses in the diaphragm holder 16, as shown in FIG. 3. Preferably, at least on of the components of the device, such as the top or the bottom or the diaphragm holder, is coated with parylene.

When the top 18 and the bottom 20 are connected together, the diaphragm holder 16 is compressed between them. The compression of the diaphragm holder 16 imparts a decompression force on the top 18 and the bottom 20 that tends to separate them, but which does not automatically occur because the top 18 and the bottom 20 are selectively locked together by the locking recess 30 and the locking ridge 50. The top 18 and the bottom 20 can be separated by spreading the top side wall 26 outwardly from the locking ridge 50 to allow the locking ridge 50 to disengage from the locking recess 30.

The diaphragm 14 is positioned within the housing 12, preferably between the top 18 and the bottom 20 to form an inlet fluid reservoir 62 and an outlet fluid reservoir 64. The diaphragm 14 is preferably concentrically aligned with the top 18, the diaphragm holder 16, and the bottom 20. When the top 18 and the bottom 20 are locked together, the protrusion 28 contacts the middle 58 of the diaphragm 14, thus biasing the diaphragm 14 towards the outlet fluid reservoir 64. The bias causes the sealing edge 56 of the diaphragm 14 to sealingly engage the annular ledge 52. In this manner, the diaphragm holder 16 holds the diaphragm 14 within the housing 12. While the sealing edge 56 is sealingly engaged against the annular ledge 52, the middle 58 of the diaphragm 14 remains flexible to alternatively move towards the fluid outlet 34 and the fluid inlet 24, as described below in operation of flow regulator device 10. Briefly, as the middle 58 of the diaphragm 14 moves towards the fluid outlet 34, the middle 58 forms an generally arcuate shape. The arcuate shape has a radius of curvature that increases as the middle 58 moves closer to the fluid outlet 34.

The fluid flow path through an embodiment of the fluid flow regulator device 10 is shown in FIG. 8. The fluid enters fluid flow regulator 10 through the inlet fluid passage 27 in the fluid inlet 24. The fluid flows from the inlet fluid passage 27 into the inlet fluid reservoir 62, then through the top fluid channel 31 in the top 18 to the capillary groove 29. From the capillary groove 29, the fluid flows to the bypass fluid channel 54 in the diaphragm holder 16, and then through a bottom fluid channel 70 between the diaphragm holder 16 and the bottom 20. The fluid then flows from the bottom fluid channel 70 to the outlet fluid reservoir 64, and finally through the outlet fluid passage 44 in the fluid outlet 34.

The flow channels 31, 54 and 70 are sufficiently large enough to allow fluids to flow through channels 31, 54 and 70 relatively unrestricted. The capillary groove 29, however, is sufficiently small enough to restrict fluid flow. Preferably, the size of the capillary groove 29 varies from a relatively large groove, having a large width and depth, to a relatively small groove, having a small width and depth. Because the capillary groove 29 restricts fluid flow, a fluid pressure drop occurs as fluid flows through the capillary groove 29. The amount of the pressure drop, and thus the flow rate through the regulator 10, can be controlled by varying the effective length of capillary groove 29.

The effective length of capillary groove 29 is the length of capillary groove 29 that fluid must flow through to enter bypass fluid channel 54. The effective length of capillary groove 29 can be equal to or less than the entire length of the capillary groove 29. The effective length of the capillary groove 29 can be varied by rotating the top 18 in relation to the diaphragm holder 16 and the bottom 20. When the top 18 rotates relative to the diaphragm holder 16, the capillary groove 29 rotates to connect the bypass fluid channel 54 to the capillary groove 29. The fluid flows through the effective length of capillary groove 29 to enter the bypass fluid channel 54 at the connection location. Therefore, the connection location along the length of the capillary groove 29 determines the effective length of capillary groove 29, the resulting fluid pressure drop, and the resulting fluid flow rate through regulator device 10.

In another embodiment of the present invention, the fluid flow regulator comprises at least two, or preferably a plurality, of ribs 48. The ribs 48 are disposed on the raised portion 38, as is shown in FIG. 3. The ribs 48 are disposed on the raised portion 38 of the bottom wall 32 to prevent the diaphragm 14 from slipping off of or tucking under the annular ledge 52 of the diaphragm holder 16 when the diaphragm 14 moves into the outlet fluid reservoir 64. The ribs 48 are connected to and extend upwardly from the raised portion 38. The ribs 48 extend into the outlet fluid reservoir 64 towards the diaphragm 14. Although FIG. 3 shows six ribs 48 on the raised portion 38, the number of ribs 48 can be increased or decreased. The number of ribs is preferably sufficient to prevent the diaphragm 14 from slipping off of or tucking under any portion of the annular ledge 52. In the embodiment shown, each rib 48 comprises two upstanding rib columns 72 spaced apart by a column space 74. One alternative rib 48 configuration would include a single rib column 72 rather than two rib columns 72 spaced apart by column space 74.

FIG. 4 shows a perspective top view of the bottom 20 of the fluid flow regulator 10 depicted in FIG. 3. The ribs 48 are symmetrically positioned on the raised portion 38 surrounding the outlet opening 46. Particularly, the ribs 48 are located at a constant radial distance from the center of the outlet opening 46. The radial distance from the center of the outlet opening 46 is short enough so that the ribs 48 do not interfere with the diaphragm holder 16 when the flow regulator device 10 is assembled. The ribs 48 preferably do not touch the diaphragm holder 16, and thus do not interfere with the compression of the diaphragm holder 16. The ribs 48 are also spaced at equal arcuate angles around the center of the outlet opening 46. The ribs 48 may, however, be positioned asymmetrically, including various radial distances and various arcuate angles, on the raised portion 38.

FIG. 5 shows an enlarged top view of a portion of the bottom 20 with a rib 48 shown in greater detail. The two upstanding rib columns 72 of the rib 48 are spaced apart by column space 74. A rib 48 constructed of two, relatively thin rib columns 72 is preferred over a single, relatively wider rib column 72. The two rib column 72 structure provides fluid flow through column space 74. The rib 48 has a rib front 76 facing the outlet opening 46 and a rib back 78 facing away from the outlet opening 46. The rib front 76 and rib back 78 have arcuate profile shapes, as shown in FIG. 5. The arcuate profile shapes have a radial center located at the center of outlet opening 46.

FIG. 6 shows an enlarged side view of a rib 48 taken along line 6-6 from FIG. 4. FIG. 6 further shows a portion of the bottom 20 in cross-section. Each rib 48 has a rib top 80 and a pair of rib sides 82. The rib top 80 connects the rib front 76, rib back 78, and rib sides 82 together. The junction of the rib top 80 with the rib back 78 is preferably a rounded corner 84. The rounded corner 84 extends into the outlet fluid reservoir 64 relatively further than the raised portion 38 at outlet opening 46. The junction of the rib top 80 with the rib front 76 extends into the outlet fluid reservoir 64 a lesser relative distance than the junction of the rib top 80 with the rib back 78. The rib top 80 thus slopes downwardly from the rib back 78 towards the rib front 76.

As shown in FIG. 6, the profile shape of the sloping rib top 80 is curved or arcuate. The arcuate shape of the rib top 80 is spaced, preferably 0.02 inches, from the diaphragm 14 when the diaphragm 14 extends into the outlet fluid reservoir 64 far enough to contact and close the outlet opening 46. The rib 48 extends into the outlet fluid reservoir 64 a predetermined distance so that the rib 48 does not contact the diaphragm 14 under normal operating conditions as described below. Likewise, the diaphragm 14 contacts the rib 48 when the diaphragm 14 moves into the outlet fluid reservoir 64 a predetermined distance.

FIG. 7 shows a cross-sectional view of a rib 48 taken along line 7-7 of FIG. 4. Particularly, FIG. 7 shows the profile shape of the column space 74 between rib columns 72. The profile of the column space 74 has a semicircular shape at the bottom where the rib 48 is attached to the raised portion 38 of the bottom wall 32 of the bottom 20.

In operation of fluid flow regulator device 10, the regulator 10 is connected to an I.V. administration set. The I.V. administration set includes an I.V. bag containing medical fluid. The I.V. bag is connected to fluid inlet 24 by I.V. tubing. The fluid outlet 34 is connected to another piece of I.V. tubing, which is in turn connected to an I.V. needle suitable for insertion into a patient's venous system. The I.V. set may include other I.V. components, for example, a drip chamber or a Y-type injection site.

The fluid flow regulator 10 is adjusted to set the desired fluid flow rate by rotating the top 18 in relation to the diaphragm holder 16 and the bottom 20. The medical fluid flows under the force of gravity from the I.V. bag to the fluid flow regulator device 10. In accord with the fluid path described above, the medical fluid enters the fluid flow regulator device 10 through the inlet fluid passage 27 in the fluid inlet 24. The medical fluid next flows through the inlet fluid passage 27 to the inlet fluid reservoir 62. The medical fluid contained in the inlet fluid reservoir 62 has an inlet fluid pressure. The fluid flows around the diaphragm 14 by flowing through the bypass fluid channel 54 in the diaphragm holder 16. More specifically, the fluid flows around the diaphragm 14 by flowing through the top fluid channel 31, the bypass fluid channel 54, and the bottom fluid channel 70. The fluid flows into the outlet fluid reservoir 64 from the bottom fluid channel 70. The fluid contained in the outlet fluid reservoir 64 has an outlet fluid pressure. The fluid flows from the outlet fluid reservoir 64 through the outlet fluid passage 44 in the fluid outlet 34. The fluid then flows from the fluid outlet 34 through the I.V. tubing and into the patient.

As the fluid flows from the inlet fluid reservoir 62 through the capillary groove 29 and to the outlet fluid reservoir 64, a fluid pressure drop occurs. Those of ordinary skill in the art may use known fluid dynamics analysis techniques to quantify the pressure drop. Because of the pressure drop, a pressure differential is created between the inlet fluid reservoir 62 and the outlet fluid reservoir 64. The outlet fluid pressure in the outlet fluid reservoir 64 is less than the inlet fluid pressure in the inlet fluid reservoir 62. This pressure differential causes the flexible diaphragm 14 to flex or move into the outlet fluid reservoir 64. Particularly, the middle 58 of the diaphragm 14 moves into the outlet fluid reservoir 64.

As the diaphragm 14 moves into the outlet fluid reservoir 64, the diaphragm 14 moves closer to the outlet opening 46 of the outlet fluid passage 44. Fluid flow through the outlet opening 46 is restricted and reduced as the diaphragm 14 approaches the outlet opening 46. The diaphragm 14 may contact the bottom 20 at the outlet opening 46 to close the outlet opening 46. Fluid flow through the flow regulator device 10, and particularly through the capillary groove 29 is reduced in relation to the reduction of fluid flow through the outlet opening 46. Because the fluid flow rate through the capillary groove 29 is reduced, the pressure drop through the capillary groove 29 is also reduced. The pressure differential between the inlet fluid reservoir 62 and the outlet fluid reservoir 64 is also, accordingly, reduced. Due to the reduced fluid pressure differential, the diaphragm 14 moves away from the outlet opening 46 and back towards the inlet fluid reservoir 62. In this sense, the diaphragm 14 is adapted to regulate the flow of fluid in the fluid passage in response to the rotation of the top 18 to the bottom 20. In one embodiment, the rate of fluid flowing through the device is regulated by the diaphragm 14 in combination with the effective length of the capillary groove 29.

The fluid flow rate through the flow regulator device 10 will increase as the diaphragm 14 moves away from the outlet opening 46 because flow through the outlet opening 46 is less restricted by the diaphragm 14. The pressure differential and corresponding fluid flow rate will change repetitively, as described above, until an equilibrium flow rate is established. The equilibrium flow rate is established relatively quickly such that the process of establishing equilibrium fluid flow does not adversely effect administration of the fluid to the patient.

The inlet fluid pressure may change due to various circumstances. For example, the inlet fluid pressure will decrease over time as the amount of fluid in the I.V. bag decreases. Also, the height of the I.V. bag above the patient may be changed. These fluid pressure changes can be measured by the amount of head height above the regulator. As the fluid inlet pressure changes, the fluid flow regulator 10 compensates for the pressure change by establishing an equilibrium as described above. A fluid flow regulator device 10 constructed in accordance with the present invention has been found to maintain average fluid flow rates within plus or minus ten percent variation despite head height movement between about thirty and about sixty inches. In this regard, the fluid flow regulator 10 maintains a constant fluid flow rate through the device.

The fluid flow regulator device 10 may be misused by injecting a bolus injection of supplementary fluid medication in the I.V. set upstream of the regulator device 10. The bolus injection of fluid may cause an extreme pressure increase in the inlet fluid reservoir. The extreme pressure increase can be compounded by repeated, forceful upstream bolus injections. The pressure increase will cause the flexible diaphragm 14 to move into the outlet fluid reservoir 64. Under normal use, the regulator device 10 would compensate for the increased pressure. The extreme pressure increase, however, may move the diaphragm 14 far into the outlet fluid reservoir 64 and, if not for the ribs 48, cause the diaphragm 15 to slip off of or tuck under the annular ledge 52. Under the extreme pressure, the diaphragm 14 moves into contact with the ribs 48, which prevents further movement of the diaphragm 14 into outlet fluid reservoir 64. The ribs 48 are positioned on the bottom 20 and extend into the outlet fluid reservoir 64 such that the diaphragm 14 abuts ribs 48 when the diaphragm 14 moves into the outlet fluid reservoir 64 a predetermined distance. The predetermined distance, which serves as the maximum movement of the diaphragm 14, is preferably small enough to prevent the diaphragm 14 from slipping off of or tucking under the annular ledge 52.

It will be understood that the invention may be embodied in other specific forms without departing from the spirit or central characteristics thereof. The present examples and embodiments, therefore, are to be considered in all respects as illustrative and not restrictive, and the invention is not to be limited to the details given herein.

1. A device for regulating the flow of intravenous fluid comprising:
a top having an inlet;
a bottom having an outlet;
wherein the top and the bottom are rotatably connected and define a housing;
wherein the inlet and outlet define a fluid passage through the housing for the intravenous fluid; and
wherein at least either the top or the bottom comprises parylene.

2. The device of claim 1 wherein the device is characterized in having a medium static turning torque less than about 42 in.-oz.

3. The device of claim 2 wherein the device is characterized in having a medium dynamic turning torque, and wherein a sum of the medium turning torques is less than about 84 in.-oz.

4. The device of claim 1 wherein the parlyene is selected from the group consisting of parylene N, parylene C, and parylene D.

5. The device of claim 1 further comprising a diaphragm disposed in the housing.

6. The device of claim 5 further comprising a diaphragm holder disposed in the housing proximate to the bottom, wherein the diaphragm is adapted to be sealingly engaged to the diaphragm holder.

7. The device of claim 6 wherein the diaphragm holder further comprises parylene.

8. The device of claim 7 wherein the device is characterized in having a medium dynamic turning torque and a medium static turning torque, and wherein a sum of the medium turning torques is less than about 84 in.-oz.

9. The device of claim 7 wherein the sum of the medium turning torques is less than about 61 in.-oz.

10. A device for regulating the flow of intravenous fluid comprising:
a top having an inlet;
a bottom having an outlet;
wherein the top and the bottom are rotatably connected and define a housing;
wherein the inlet and outlet define a fluid passage through the housing for the intravenous fluid;
a diaphragm holder disposed in the housing; and
wherein at least either the top or bottom or the diaphragm holder comprises parylene.

11. The device of claim 10 wherein the device is characterized in having a medium static turning torque less than about 42 in.-oz.

12. The device of claim 11 wherein the device is characterized in having a medium dynamic turning torque, and wherein a sum of the medium turning torques is less than about 84 in.-oz.

13. The device of claim 10 wherein the parlyene is selected from the group consisting of parylene N, parylene C, and parylene D.

14. The device of claim 10 further comprising a diaphragm disposed in the housing and adapted to be sealingly engaged to the diaphragm holder

15. The device of claim 14 wherein the diaphragm holder comprises parylene.

16. The device of claim 15 wherein the device is characterized in having a medium dynamic turning torque and a medium static turning torque, and wherein a sum of the medium turning torques is less than about 84 in.-oz.

17. The device of claim 10 further comprising a channel disposed in the diaphragm holder.

18. The device of claim 10 wherein the parylene has a thickness of about 0.10 microns to about 3.0 microns
